(19) United States
(12) Patent Application Publication
(10) Pub. No.: US 2007/0112376 A1
(43) Pub. Date: May 17, 2007

(54) Medical tubing clamping apparatus

(75) Inventor: Donald J. Propp, Dewitt, MI (US)

Correspondence Address: Christopher J. Fildes;Fildes & Outland, P.C., Suite 2, 20916 Mack Avenue, Grosse Pointe Woods, MI, 48236, US

(73) Assignee: Tri-State Hospital Supply Corporation

(21) Appl. No.: 11/273,068

(22) Filed: Nov. 14, 2005

(51) Int. Cl. A61B 17/00

(52) U.S. Cl. 606/205

(57) ABSTRACT

A medical tubing clamping apparatus includes a first elongated member having a handle end portion and a working end portion, and a second elongated member having a handle end portion and a working end portion. The first elongated member is pivotally connected to the second elongated member between the handle end portion and the working end portion. The first member working end portion and the second member working end portion are pivotal between an open position and a closed position. The first member working end portion and the second member working end portion are spaced in the closed position and define a gap therebetween. In the closed position, the working portions can clamp medical tubing to prevent flow of fluid through the tubing without damaging the tubing.

### TECHNICAL FIELD

This invention relates to medical instruments, and more particularly to medical tubing clamping apparatus for clamping medical tubing.

### BACKGROUND OF THE INVENTION

It is known in the medical field that patients brought to a CT scanning department for a CT scan may have an operational pump or gravity drip IV fluid administration set connected by medical tubing to an intravenous catheter. The CT scan often requires high pressure contrast media (dye) injection through the IV catheter. In order to inject the contrast media through the catheter, it may become necessary to disconnect the IV administration set, or extension set, from the catheter. Even if the IV administration set or extension set has a Y-valve or port on it, the tubing of the IV administration set is usually not designed for high pressure applications (in this case, high pressure is considered to be in the general range of 50-500 psi). Further, any snap clamps, slide clamps, or anti-reflux check valves that may be located above the Y-valve are not usually designed for high pressure applications and are not sufficient to stop the flow of fluid under high pressure. Thus, it is conventionally necessary to remove the IV administration set, install a high pressure set, and after the contrast media injection and CT scan, remove the high pressure set and reattach an ordinary “low pressure” IV extension or IV administration set as necessary. This procedure includes a costly number of steps and is also a significant disturbance to the patient.

Further, even though it is not difficult to make low cost high pressure tubing sets, there are no tubing slides, snap clamps, or inline anti-reflux flow check valves in existence that are effective for both high pressure lines and ordinary low pressure lines. Thus, even if a high pressure application Y-port were built into an ordinary IV extension or IV administration set, it still could not be used unless the IV line from the IV pump or bag connected to the straight inlet of the Y-port is removed and dead end capped prior to dye injection into the Y-port. To do otherwise might send dye to the IV pump, which could cause damage, or might cause the low pressure IV bag to burst.

It is also known to temporarily clamp off tubing with hemostats, forceps, or needle holders. These devices, however, usually have teeth or jaws with abrupt and/or sharp corners as well as non-smooth geometries on the gripping surface to prevent the sliding or slipping of an object held by the jaws under pull forces. These non-smooth surfaces may cut the tubing. Further, these devices provide non-uniform, uncontrolled pressure on the tubing wall. If these devices are used on high pressure IV tubing at even the low end of the CT scan pressure range, the non-smooth surfaces and sharp edges of the jaws combined with tubing swelling, can lead to tubing wall damage or rupture due to radial shear forces. Further, these devices have a tendency to permanently deform the tubing, causing the tubing to not reopen, or to be quite flow restrictive, after release of the clamps.

Therefore, a need exists for a clamping apparatus that can temporarily (for a few minutes) clamp shut a high pressure IV tubing set having high pressure tubing walls without damaging the tubing.

### SUMMARY OF THE INVENTION

The present invention provides a medical tubing clamping apparatus that prevents flow of fluid through medical tubing when clamped on the tubing while also avoiding cutting, crushing, deforming, or otherwise damaging the tubing. The clamping apparatus can successfully block the flow of fluid during high pressure applications, such as contrast media injection, without damaging the tubing. This allows for the continued use of the tubing after clamping with the clamping apparatus.

More particularly, a medical tubing clamping apparatus in accordance with the present invention includes a first elongated member having a handle end portion and a working end portion and a second elongated member having a handle end portion and a working end portion. The first elongated member is pivotally connected to the second elongated member between the handle end portion and the working end portion. The first member working end portion and the second member working end portion are pivotal between an open position and a closed position, and the first member working end portion and second member working end portion are spaced in the closed position and define a gap therebetween. In the closed position the working portions clamp medical tubing to prevent flow of fluid through the tubing without damaging the tubing.

The gap may be generally continuous in width. An inner surface of the first and second member working end portions may be a flat, tube engaging surface. The inner surfaces may include transversely extending grooves in said inner surfaces distal a tip of the working end portions. The grooves may be cooperatively adjacent each other in the closed position.

Optionally, the first and second member working end portions may be generally free of sharp edges. Each of the first and second handle end portions may include a circular handle and a locking mechanism adjacent the handle. The first elongated member and second elongated member may be pivotally connected by a box hinge or any other suitable pivoting arrangement.

In a specific embodiment, a medical tubing clamping apparatus in accordance with the present invention includes a first elongated member and a second elongated member. The first elongated member includes a handle at an end thereof and a jaw at an opposite end, and the second elongated member also includes a handle at an end thereof and a jaw at an opposite end. The first and second elongated members are pivotally connected to each other between the ends, and the jaws are thereby pivotal between an open position and a closed position. In the closed position, the jaws are spaced from each other, defining a gap, and the jaws may thereby clamp medical tubing to prevent flow of fluid through the tubing without damaging the tubing.

Each of the jaws may include a generally flat inner surface, and the flat inner surfaces may be generally parallel in the closed position. The gap may be between approximately 0.01 and 0.03 inches in width, and the width of the gap may be generally constant. Each of said jaws may also include a transversely extending groove in said inner surfaces distal a tip thereof. The first and second elongated members may include a cooperating locking mechanism for locking the jaws in the closed position. The first and second elongated members may be pivotally connected by a box hinge or similarly suitable pivotal connection.

These and other features and advantages of the invention will be more fully understood from the following detailed description of the invention taken together with the accompanying drawings.

### BRIEF DESCRIPTION OF THE DRAWINGS

In the drawings:

FIG. 1 is a side view of a medical tubing clamping apparatus in accordance with the present invention illustrating the clamping apparatus in an open position;

FIG. 2 is a side view of the medical tubing clamping apparatus of FIG. 1 illustrating the clamping apparatus in a closed position;

FIG. 3 is an enlarged partial view of the medical tubing clamping apparatus of FIG. 2 illustrating a working end of the apparatus;

FIG. 4 is a perspective view of the medical tubing clamping apparatus of FIG. 1; and

FIG. 5 is an environmental view illustrating the medical tubing clamping apparatus of FIG. 1 clamping high pressure medical tubing of a high pressure extension set.

### DETAILED DESCRIPTION OF THE INVENTION

Referring now to the drawings in detail, numeral 10 generally indicates a medical tubing clamping apparatus in accordance with the present invention that allows for the temporary clamping shut of high pressure medical tubing without damaging the tubing. For example, the clamping apparatus 10 may be used to clamp shut a length of high pressure tubing above a Y-valve port in a high pressure extension set during high pressure (50-500 psi) injection of fluid (e.g., contrast media) through the tubing. The clamping apparatus 10 successfully blocks the flow of fluid in this high pressure range. Further, since the clamping apparatus 10 does not cut, crush, or deform the tubing, the tubing may be continually used after release of the clamping apparatus without the need for replacement of the tubing/extension set. This reduces costs in terms of supplies and labor, as well as improves patient comfort by eliminating the need for the patient to undergo an extension set replacement procedure.

Further, the clamping apparatus 10 reduces the risk of IV pump or bag damage due to back flow of high pressure fluid, eliminating the need to disconnect and cap off running IV lines above the Y-valve port, or to cap off any inlets above the Y-valve port. The clamping apparatus 10 also reduces the need to abort high pressure injection runs because of tubing or clamp failure. Finally, the clamping apparatus 10 is reusable and does not have to be included as part of a disposable extension set kit or IV administration kit. This can reduce the cost of disposable kits that conventionally require some form of clamp or dead end cap, and can also eliminate the need for separate dead end cap kits.

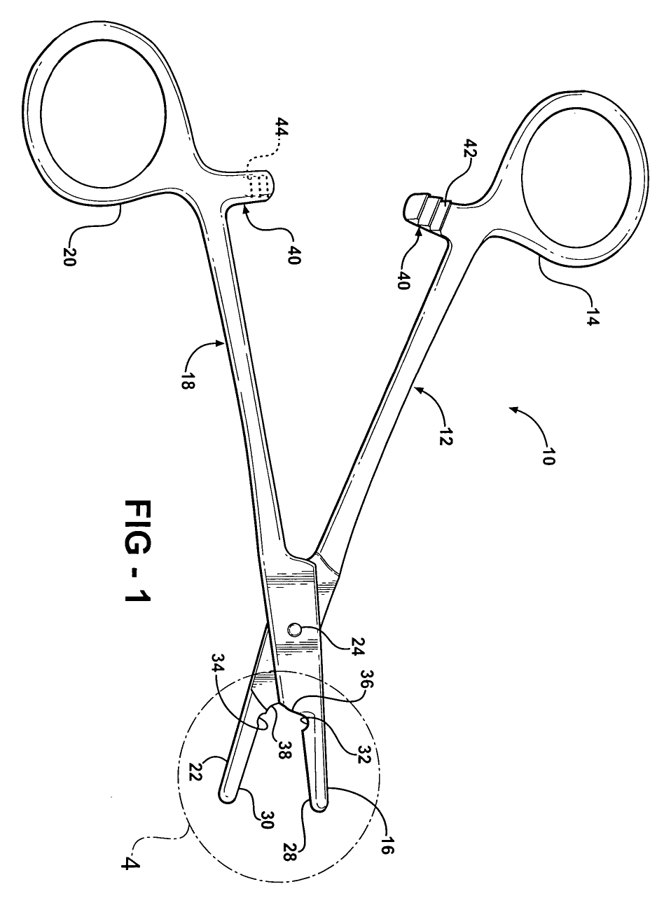

With reference to FIGS. 1 through 4, a medical tubing clamping apparatus in accordance with the present invention includes a first elongated member 12 having a handle end portion 14 and an opposite working end portion 16. The handle end portion 14 may be a handle, such as a circular handle or any other handle design suitable for engagement with a user's fingers. The working end portion 16 may be a jaw or similar. The clamping apparatus 10 further includes a second elongated member 18 having a handle end portion 20 and an opposite working end portion 22. The handle end portion 20 may be a handle, such as a circular handle or similar, and the working end portion 22 may be a jaw or similar. The second elongated member 18 may be similar to, but not necessarily identical to, the first elongated member 12, and the two elongated members 12, 18 are cooperable as is described in more detail below. The medical tubing clamping apparatus 10 may preferably be made of metal, but may also be made of plastic or any other similarly suitable material.

The first elongated member 12 and second elongated member 18 are pivotally connected to each other between the handle end portions 14, 20 and the working end portions 16, 22 at a pivot point 24. The first member working end portion 16 and the second member working end portion 22 are thereby pivotal between an open position, as shown in FIG. 1, and a closed position, as shown in FIG. 2. In the closed position, the first member working end portion 16 and second member working end portion 22 are spaced and define a gap 26 therebetween. While in the closed position, the working portions 16, 22 can clamp medical tubing, such as high pressure tubing or similar, to prevent flow of fluid through the tubing without causing damage to the tubing.

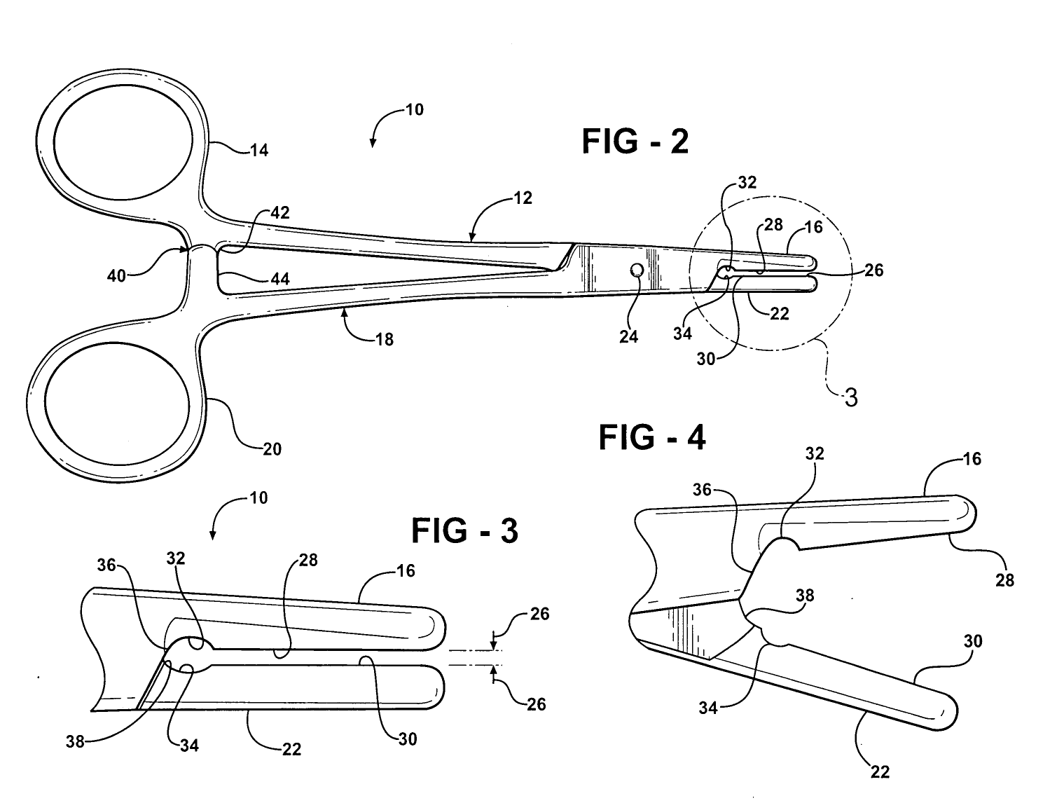

The gap 26 may be generally continuous in width, the width being defined as the distance from one working portion inner surface 28 to the other working portion inner surface 30. The width may be between approximately 0.01 and 0.03 inches. The gap may be generally designed to have a width that is approximately equal to a value of 50% of the compression of twice the tubing wall thickness. This provides for the clamping shut of tubing in the gap 26 without cutting or otherwise damaging the tubing. Likewise, the first and second member working end portions 16, 22 may each include a generally flat, tube engaging inner surface 28, 30 respectively. The flat inner surfaces 28, 30 may be generally parallel to each other in the closed position as shown particularly in FIGS. 2 and 3. The flat inner surfaces 28, 30 may be designed to have a length equal to approximately three times the width of a flattened piece of tubing. This helps to assure that a user does not clamp tubing outside of the area of the inner surfaces. The flat inner surfaces 28, 30 may also be designed to have a width that is approximately equal to the width of a flattened piece of tubing to make the surfaces 28, 30 non-cutting.

The inner surfaces 28, 30 may each include a transversely extending groove 32, 34 therein distal tips of the working portions 16, 22. The grooves 32, 34 may be cooperatively adjacent each other in the closed position. The grooves 32, 34 help to prevent tubing that is clamped at or near back ends 36, 38 of the working portions 16, 22 from being cut or severed. The first and second working portions 16, 22 may also be generally free of sharp edges, which further helps to prevent the clamping apparatus 10 from cutting or otherwise damaging medical tubing clamped in the working portions.

The first and second elongated members 12, 18 may be pivotally connected by a box hinge or any other suitable pivotal connection. For example, the first and second elongated members 12, 18 may be connected side-by-side by a pivot pin passing through both members at the pivot point 24. The pivotal connection may also be closer to the working portions 16, 22 than the handle portions 14, 20.

The medical tubing clamping apparatus 10 may also include a cooperating locking mechanism 40 for locking the working portions 16, 22 in the closed position. The first and second elongated members 12, 18 may each include a part 42, 44 of the cooperating locking mechanism 40 adjacent the handle portions 14, 20. Specifically, the locking mechanism 40 may be a locking ratchet or similarly suitable locking arrangement.

Turning now to FIGS. 1, 2, and 5, the medical tubing clamping apparatus 10 is effective to clamp shut high pressure medical tubing without damaging the tubing. The high pressure tubing may be part of a high pressure tubing extension set 46 connected to a catheter hub 48 of a catheter 50. The tubing in the extension set 46, for purposes of example, may have an outside diameter of approximately 0.109 inches, an inside diameter of 0.053 inches, a wall thickness of 0.028 inches, and a Shore A durometer of 90A. The tubing is therefore relatively rigid, making it capable of withstanding high internal pressures against the tubing inner surface. The extension set 46 may include a Y-valve port 52 connected to a main line 54. The main line may extend and connect to an IV pump or IV gravity drip bag line, while the valve port 52 may be used to inject CT scan contrast media dye at high pressures (e.g., 300 psi) for a short period of a minute or more. IV fluids are fed to the catheter 50 through the main line 54, and the catheter 50 is inserted into a patient's vein (not shown) for introduction of the fluid into the patient's bloodstream. In order to conduct a CT scan, contrast media dye must be injected into the tubing and catheter 50 via the valve port 52. This requires the main line 54 to be shut upstream of the valve port 52 to prevent the contrast media from traveling up the main line, which could damage the IV pump or burst the IV bag.

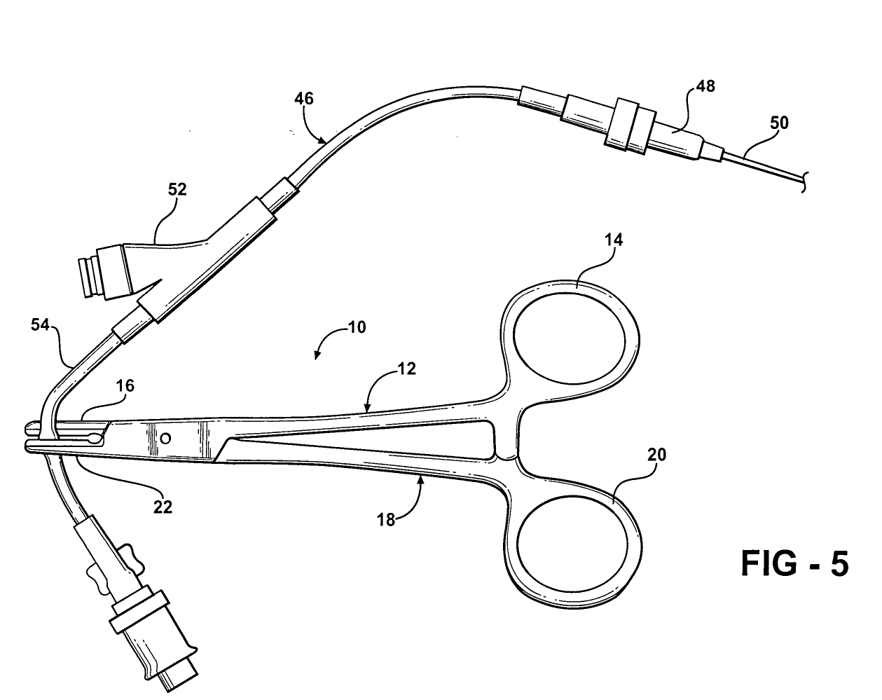

The medical tubing clamping apparatus 10 of the present invention can be used to clamp shut the high pressure tubing along the main line 54. To use the clamping apparatus 10, a user such as a nurse or other medical clinician may grasp the handle portions 14, 20 with his/her hand using, for example, the thumb and middle finger. The user then positions the working portions 16, 22 of the apparatus 10 about the tubing in the main line upstream of the Y-valve port 52 such that the tubing is in between the working portions. Next, the user squeezes the handle portions 14, 20 to close the working portions 16, 22 on the tubing as shown in FIG. 5. The locking mechanism 40 holds the working portions 16, 22 in the closed position so that the user may release the handle portions 14, 20 without the clamp coming open.

The working portions 16, 22 squeeze the circular tubing wall together, thereby flattening the tubing, so that the inside diameter of the tubing is effectively reduced to approximately zero (or slightly less than zero). This prevents the flow of fluid past this clamped portion of tubing. The gap 26 between the inner surfaces 28, 30 of the working portions 16, 22, however, assures that the clamp does not deform, crush, or cut the tubing wall when in the closed position. The gap 26 in the working portions 16, 22 is specifically designed so that sufficient pressure is exerted on the tubing wall in the closed position to seal the tubing shut. At the same time, the gap 26 prevents the working portions 16, 22 from exerting too much pressure on the tubing wall so as to prevent damage to the tubing.

Further, since the tube engaging inner surfaces 28, 30 of the working portions 16, 22 are generally flat and smooth, the working portions 16, 22 evenly press the tubing wall together. In other words, an even amount of force is applied across the tubing wall. The surfaces of the working portions 16, 22 also have generally smooth, rounded edges and are free of sharp edges and surfaces. This helps to prevent the working portions 16, 22 from cutting the tubing. The grooves 32, 34 help to prevent tubing that is clamped near or at the back ends 36, 38 of the working portions 16, 22 from being cut or severed.

With the tubing clamped shut, contrast media dye may be injected at high pressures through the valve port 52 without the contrast media dye traveling past the clamped portion of tubing. The contrast media dye travels to the catheter 50, where it enters the patient's bloodstream. After the CT scan is complete, the clinician may remove the clamping apparatus 10 by releasing the locking mechanism 40 and opening the handle portions 14, 20, which in turn opens the working portions 16, 22. The clamped portion of tubing returns to its original configuration, opening the main line 54 of tubing for flow of fluid from the IV pump, bag, or similar. There is no need to remove and replace the extension set 46, or the dressing that is usually covering the extension set catheter, saving both time and money. Further, patient comfort is improved as the patient does not have to be subjected to extension set and dressing replacement, which often also involves undressing and redressing the catheter 50.

Since the clamping apparatus 10 only contacts the outside of the IV tubing and does not come in contact with bodily fluids, it may be cleaned and disinfected after use. The cleaning procedure may be conducted directly in the CT department, or in any other location where the apparatus is used. Hence, the clamping apparatus 10 may be used over and over again without disposal. Although the invention has been described by reference to a specific embodiment, it should be understood that numerous changes may be made within the spirit and scope of the inventive concepts described. Accordingly, it is intended that the invention not be limited to the described embodiment, but that it have the full scope defined by the language of the following claims.

What is claimed is:

1. A medical tubing clamping apparatus comprising:
a first elongated member having a handle end portion and a working end portion; and
a second elongated member having a handle end portion and a working end portion;
said first elongated member being pivotally connected to said second elongated member between said handle end portion and said working end portion;
said first member working end portion and said second member working end portion being pivotal between an open position and a closed position;
wherein said first member working end portion and said second member working end portion are spaced in said closed position and define a gap therebetween;
whereby in said closed position said working portions clamp medical tubing to prevent flow of fluid through said tubing without damaging said tubing.

2. The medical tubing clamping apparatus of claim 1, wherein said gap is generally continuous in width.

3. The medical tubing clamping apparatus of claim 1, wherein said first and second member working end portions each include a generally flat tube engaging inner surface.

4. The medical tubing clamping apparatus of claim 3, wherein said inner surface of said first and second member working end portions include a transversely extending groove in said inner surfaces distal a tip of said working end portions.

5. The medical tubing clamping apparatus of claim 4, wherein said grooves are cooperatively adjacent each other in said closed position.

6. The medical tubing clamping apparatus of claim 1, wherein said first and second member working end portions are generally free of sharp edges.

7. The medical tubing clamping apparatus of claim 1, wherein each of said first and second handle end portions includes a circular handle and a locking mechanism adjacent said handle.

8. The medical tubing clamping apparatus of claim 1, wherein said first elongated member and second elongated member are pivotally connected by a box hinge.

9. A medical tubing clamping apparatus comprising:
a first elongated member; and
a second elongated member;
said first elongated member including a handle at an end thereof and a jaw at an opposite end;
said second elongated member including a handle at an end thereof and a jaw at an opposite end;
said first and second elongated members being pivotally connected to each other between said ends;
wherein said jaws are pivotal between an open and closed position, and in said closed position, said jaws are spaced from each other and define a gap;
whereby in said closed position said jaws clamp medical tubing to prevent flow of fluid through said tubing without damaging said tubing.

10. The medical tubing clamping apparatus of claim 9, wherein each of said jaws includes a generally flat inner surface.

11. The medical tubing clamping apparatus of claim 10, wherein said flat inner surfaces of said jaws are generally parallel in said closed position.

12. The medical tubing clamping apparatus of claim 9, wherein said gap is between approximately 0.01 and 0.03 inches in width.

13. The medical tubing clamping apparatus of claim 12, wherein said width of said gap is generally constant.

14. The medical tubing clamping apparatus of claim 9, wherein each of said jaws includes a transversely extending groove in said inner surfaces distal a tip thereof.

15. The medical tubing clamping apparatus of claim 9, wherein said first and second elongated members include a cooperating locking mechanism for locking said jaws in said closed position.

16. The medical tubing clamping apparatus of claim 9, wherein said first elongated member and second elongated member are pivotally connected by a box hinge.
